(19) United States
(12) Patent Application Publication
(10) Pub. No.: US 2023/0157879 A1
(43) Pub. Date: May 25, 2023

(54) GENERATING BUBBLE JETS TO FRAGMENT AND REMOVE EYE FLOATERS

(71) Applicant: Alcon Inc., Fribourg (CH)

(72) Inventors: Zsolt Bor, San Clemente, CA (US); Alireza Malek Tabrizi, Fremont, CA (US); Raul Mauricio, Mission Viejo, CA (US)

(21) Appl. No.: 18/049,215

(22) Filed: Oct. 24, 2022

Related U.S. Application Data
(60) Provisional application No. 63/281,356, filed on Nov. 19, 2021.

(51) Int. Cl. A61F 9/008

(52) U.S. Cl. CPC A61F 9/008; A61B 2017/22007; A61F 2009/00874

(57) ABSTRACT

In certain embodiments, an ophthalmic laser system for treating a floater in a vitreous of an eye includes a laser device that directs laser pulses towards the floater to yield cavitation bubbles that create a bubble jet to treat the floater. In some examples, the laser device includes a beam multiplexer that splits a laser beam into multiple beams that form the cavitation bubbles that create the bubble jet. In some examples, the laser device directs laser pulses towards the floater according to a pulse pattern that forms the cavitation bubbles that create the bubble jet.

### TECHNICAL FIELD

The present disclosure relates generally to laser vitreolysis systems, and more particularly to generating bubble jets to fragment and remove eye floaters.

### BACKGROUND

Eye floaters are microscopic collagen fibers that can clump and cast shadows on the retina, which disturb the vision of the patient. In laser vitreolysis, a laser beam is directed into the vitreous to treat eye floaters. The laser beam may be used to disintegrate the floaters to improve vision.

### BRIEF SUMMARY

In certain embodiments, an ophthalmic laser system for treating a floater in a vitreous of an eye comprises a laser device, an ophthalmic microscope, and a computer. The laser device directs laser pulses towards the floater in the vitreous of the eye. The laser device includes a laser that generates a laser beam, and a beam multiplexer that splits the laser beam into beams that form cavitation bubbles to create a bubble jet in the vitreous of the eye. The ophthalmic microscope provides an image of a shadow cast by the floater onto a retina of the eye. The computer instructs the laser device to direct the beams towards the floater in the vitreous in order to create the bubble jet to treat the floater.

Embodiments may include none, one, some, or all of the following features:
The beam multiplexer comprises an optical device selected from the following: a diffractive optical element (DOE), a holographic optical element (HOE), a spatial light modulator (SLM), a polarizing prism, a beam amplitude splitting interferometer, a wavefront splitting interferometer, or a birefringent optical component.
The beam multiplexer includes a wave plate that alters a polarization state of the laser beam, and a prism that separates the laser beam into the beams. The wave plate may be a half-wave plate that shifts the polarization state of the laser beam. The prism may be a Wollaston prism that separates the laser beam into the beams with orthogonal polarization.
The beam multiplexer splits the laser beam into the beams that form the cavitation bubbles with a bubble center separation of 5 to 20 microns.
The beam multiplexer creates a first cavitation bubble with a first diameter and a second cavitation bubble with a second diameter, where the second diameter is different from the first diameter.
The computer instructs the laser device to direct the beams to form the cavitation bubbles arranged to direct the bubble jet in a particular direction.
The computer instructs the laser device to direct the beams to form the cavitation bubbles arranged in a spiral enface pattern.
The computer instructs the laser device to direct the beams to form the cavitation bubbles arranged in a raster enface pattern.

In certain embodiments, a method for treating a floater in a vitreous of an eye comprises generating a laser beam by a laser of a laser device. The laser beam is split, by a beam multiplexer of the laser device, into beams that form cavitation bubbles to create a bubble jet in the vitreous of the eye. An image of a shadow cast by the floater onto a retina of the eye is provided by an ophthalmic microscope. The laser device is instructed by a computer to direct the beams towards the floater in the vitreous in order to create the bubble jet to treat the floater. The beams are directed by the laser device towards the floater in the vitreous of the eye.

Embodiments may include none, one, some, or all of the following features:
The beam multiplexer comprises an optical device selected from the following: a diffractive optical element (DOE), a holographic optical element (HOE), a spatial light modulator (SLM), a polarizing prism, a beam amplitude splitting interferometer, a wavefront splitting interferometer, or a birefringent optical component.
The method further comprises altering, by a wave plate of the beam multiplexer, a polarization state of the laser beam; and separating, by a prism of the beam multiplexer, the laser beam into the beams. The wave plate may be a λ/2 wave plate that shifts the polarization state of the laser beam, and the prism may be a Wollaston prism that separates the laser beam into the beams with orthogonal polarization.
Splitting the laser beam comprises splitting the laser beam into the beams that form the cavitation bubbles with a bubble center separation of 5 to 20 microns.
The method further comprises creating, by the beam multiplexer, a first cavitation bubble with a first diameter; and creating, by the beam multiplexer, a second cavitation bubble with a second diameter, where the second diameter is different from the first diameter.
Instructing the laser device to direct the beams towards the floater comprises instructing the laser device to direct the beams to form the cavitation bubbles arranged to direct the bubble jet in a particular direction.
Instructing the laser device to direct the beams towards the floater comprises instructing the laser device to direct the beams to form the cavitation bubbles arranged in a spiral enface pattern.
Instructing the laser device to direct the beams towards the floater comprises instructing the laser device to direct the beams to form the cavitation bubbles arranged in a raster enface pattern.

In certain embodiments, an ophthalmic laser system for treating a floater in a vitreous of an eye comprises a laser device, a floater detection system, and a computer. The laser device directs laser pulses towards the floater in the vitreous of the eye. The floater detection system detects the floater in the vitreous. The computer accesses a pulse pattern for the laser pulses, where the pulse pattern yields cavitation bubbles that create a bubble jet in the vitreous of the eye. The computer instructs the laser device to direct the laser pulses towards the floater according to the pulse pattern to create the bubble jet to treat the floater.

Embodiments may include none, one, some, or all of the following features:
The computer instructs the laser device to: create a first cavitation bubble with a first diameter, and create a second cavitation bubble with a second diameter, where the second diameter is different from the first diameter.
The computer instructs the laser device to direct the laser pulses to form the cavitation bubbles arranged to direct the bubble jet in a particular direction.
The pulse pattern yields the cavitation bubbles with a bubble center separation of 5 to 20 microns.
The pulse pattern comprises pulse groups, where each pulse group yields a set of cavitation bubbles that form a bubble jet.
The pulse pattern yields the cavitation bubbles arranged in a spiral enface pattern.
The pulse pattern yields the cavitation bubbles arranged in a raster enface pattern.
The floater detection system configured to determine a location of the floater in the vitreous of the eye.
The laser device includes a laser that generates a laser beam and a beam multiplexer that splits the laser beam into the laser pulses that form the cavitation bubbles to create the bubble jet.
The ophthalmic laser system includes an xy-scanner that: receives a detection beam from the floater detection system and directs the detection beam along a detection beam path towards a floater shadow cast by the floater on a retina of the eye; and receives the laser pulses from the laser device and directs the laser pulses along the detection beam path towards the floater shadow.

In certain embodiments, a method for treating a floater in the vitreous of an eye comprises detecting the floater by a floater detection system. A pulse pattern for laser pulses is accessed by a computer, where the pulse pattern yields cavitation bubbles that create a bubble jet in the vitreous. A laser device is instructed by the computer to direct the laser pulses towards the floater according to the pulse pattern to create the bubble jet to treat the floater.

Embodiments may include none, one, some, or all of the following features:
Instructing the laser device to direct the laser pulses towards the floater comprises instructing the laser device to create a first cavitation bubble with a first diameter and create a second cavitation bubble with a second diameter, the second diameter different from the first diameter.
Instructing the laser device to direct the laser pulses towards the floater comprises instructing the laser device to direct the laser pulses to form the cavitation bubbles arranged to direct the bubble jet in a particular direction.
The pulse pattern yields the cavitation bubbles with a bubble center separation of 5 to 20 microns.
The pulse pattern comprises pulse groups, where each pulse group yields a set of cavitation bubbles that form a bubble jet.
The pulse pattern yields the cavitation bubbles arranged in a spiral enface pattern.
The pulse pattern yields the cavitation bubbles arranged in a raster enface pattern.
The method further comprises determining, by the floater detection system, the location of the floater.
The method further comprises generating, by a laser of the laser device, a laser beam; and splitting, by a beam multiplexer of the laser device, the laser beam into the laser pulses that form the cavitation bubbles to create the bubble jet.
The method further comprises: receiving, by an xy-scanner, a detection beam from the floater detection system and directing the detection beam along a detection beam path towards a floater shadow cast by the floater on a retina of the eye; and receiving, by the xy-scanner, the laser pulses from the laser device and directing the laser pulses along the detection beam path towards the floater shadow.

### BRIEF DESCRIPTION OF THE DRAWINGS

FIG. 1 illustrates a simplified example of an ophthalmic system that creates bubble jets to fragment and remove eye floaters from an eye, according to certain embodiments;

FIGS. 2A and 2B illustrate an example of an ophthalmic laser system with a beam multiplexer that can create cavitation bubbles to form a bubble jet, according to certain embodiments;

FIG. 3 illustrates an example of an ophthalmic laser surgical system with a scanner that can create cavitation bubbles to form a bubble jet, according to certain embodiments;

FIG. 4 illustrates an example of a laser pulse causing a floater to jump;

FIG. 5 illustrates an example of a bubble jet that may be created by the system of FIGS. 2A, 2B, and 3;

FIG. 6 illustrates an example of a bubble jet 9 that may be created by the system of FIGS. 2A, 2B, and 3;

FIGS. 7A and 7B illustrate examples of a bubble jet resulting from cavitation bubbles of different diameters;

FIGS. 8 and 9 illustrate examples of enface pulse patterns that may be generated by the system of FIG. 1;

FIG. 10 illustrates an example of a method for creating a bubble j et to fragment a floater, which may be performed by the system of FIG. 1; and

FIG. 11 illustrates an example of a method for creating bubble jets to remove floater fragments, which may be performed by the system of FIG. 1.

### DESCRIPTION OF EXAMPLE EMBODIMENTS

Referring now to the description and drawings, example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

Laser vitreolysis can be performed to treat eye floaters in an eye. Current laser systems, however, fail to effectively and efficiently fragment and remove floaters, resulting in prolonged surgical time and retinal radiation exposure. To improve floater removal, ophthalmic systems described herein have a laser device that directs laser pulses towards a floater in the eye. The laser pulses form cavitation bubbles to create a bubble jet that fragments the floater and moves the floater fragments away from the visual field. In some examples, the laser device includes a beam multiplexer that splits a laser beam into multiple beams that form the cavitation bubbles that create the bubble jet. In some examples, the laser device directs laser pulses towards the floater according to a pulse pattern that forms the cavitation bubbles that create the bubble jet. In certain embodiments, laser device directs the pulses to yield a pattern (e.g., a spiral or raster pattern) of bubble jets. In the embodiments, some pulses block floater movement, reducing the likelihood the floater will jump. Accordingly, certain embodiments improve the effectiveness and efficiency of floater fragmentation and removal.

### 1. Example Systems

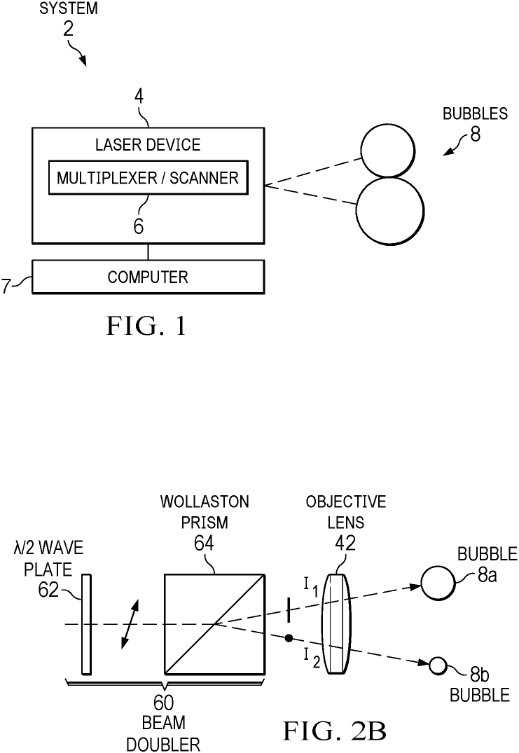

FIG. 1 illustrates a simplified example of an ophthalmic system 2 that creates bubble jets to treat, e.g., fragment and/or remove, eye floaters from an eye, according to certain embodiments. In the example, system 2 includes a laser device 4, a multiplexer and/or scanner (multiplexer / scanner) 6, and a computer 7, coupled as shown. For ease of explanation, an axis (e.g., optical or visual axis) of the eye approximates a z-axis, which in turn defines enface planes (e.g., xy-planes) substantially orthogonal to the z-axis. An enface pulse pattern (e.g., a spiral or raster enface pulse pattern) is a pulse pattern formed on an enface plane.

As an overview, laser device 4 generates a laser beam comprising laser pulses. Multiplexer / scanner 6 directs the laser pulses towards the vitreous of an eye. The laser pulses cause laser-induced optical breakdowns (LIOBs) that photodisrupt the vitreous and create rapidly expanding (and contracting) cavitation bubbles 8 that may expand and contract several times. Interaction between cavitation bubbles 8 creates a bubble jet, which is a forceful jet of water. Energy of the bubbles (such as energy of internal high-pressure gas and of surface tension forces) is converted into the kinetic energy of the bubble jet. If the bubbles are of different size, the direction of motion of the bubble jet is towards the smaller bubble. The bubble jet fragments the floater and moves the floater fragments away from the visual axis, i.e., the surgeon’s visual field.

In certain embodiments, bubbles and/or bubble jets facilitate removal of the floater fragments. For example, if the patient’s head is in an upright position during surgery, the cavitation bubbles can be oriented such that the resulting bubble jet is directed towards the upper part of the posterior chamber to move fragments away from the visual field. As another example, after floater fragmentation, residual tiny bubbles become entangled in the floater fragments, and the bubbles’ buoyancy lift the fragments away from the visual field. As another example, after a cavitation bubble repeatedly expands and collapses a few times, the water vapor in the cavitation bubble condense into water and some gases (e.g., H₂, O₂, CO₂, and NOx) remain in the bubble. These gas bubbles become entangled with the floater fragments and lift the fragments to the uppermost part of the posterior chamber, typically in about one minute. After several minutes, the gas bubbles have been absorbed into the vitreous, and the fragments have moved away from the visual field.

Turning to the components, laser device 4 may comprise any suitable ultrashort (e.g., nanosecond, picosecond, or femtosecond) pulse laser device. Examples of laser device 22 include YAG lasers (e.g., a Q-switched nanosecond YAG laser, such as a frequency doubled Q-switched nanosecond YAG laser), picosecond lasers (e.g., a mode-locked picosecond laser operating in the 1 to 1.1 micron (µm) spectral range or their second harmonics or an ultrashort infrared (700 to 1500 nanometers (nm)) picosecond laser), femtosecond lasers (e.g., an infrared, an ultrashort infrared (700 to 1500 nanometers (nm)), or ultraviolet femtosecond laser), and single pulse to high repetition rate (10 megahertz (MHZ)) lasers. The laser beam may have any suitable wavelength (e.g., 400 to 2000 nanometers (nm)) and focal point (e.g., 3 to 10 microns (µm), such as 5 to 6 microns). The pulses may have any suitable duration (e.g., 20 femtoseconds (fs) to 1000 nanoseconds (ns)), repetition rate (e.g., 25 to 100 kilohertz (kHz), such as 50 kHz), and pulse energy (e.g., 1 microjoule (µJ) to 1 millijoule (mJ), such as 1 to 20 µJ or 1 to 10 µJ).

Multiplexer / scanner 6 may comprise a multiplexer and/or scanner. A multiplexer comprises any suitable optical device that can split (or otherwise modulate) the laser beam to yield multiple laser beams, where each beam creates a cavitation bubble in the vitreous. In general, an optical device is a component that can act on (e.g., transmit, reflect, refract, diffract, collimate, condition, shape, focus, modulate, and/or otherwise act on) light. Examples of beam multiplexers include a diffractive optical element (DOE) (e.g., a diffraction grating), a holographic optical element (HOE), a spatial light modulator (SLM) (e.g., an electrically addressable SLM), a polarizing prism (e.g., a Wollaston, Normarski, Rochon, or Senamont prism), a beam amplitude splitting interferometer (e.g., a Michelson, Mach-Zender, or Fizeau wedge interferometer), a wavefront splitting interferometer (e.g., a Lloyd mirror or Fresnel biprism), a birefringent optical component, or a combination of different beam multiplexers (e.g., 5x diffractive multiplexer and a Wollaston-doubler).

A scanner moves focal point of the laser beam to different locations of an enface plane to create cavitation bubbles in the vitreous. Examples of scanners include a galvo scanner (e.g., a pair of galvanometrically-actuated scanner mirrors that can be tilted about mutually perpendicular axes), an electro-optical scanner (e.g., an electro-optical crystal scanner) that can electro-optically steer the beam, or an acousto-optical scanner (e.g., an acousto-optical crystal scanner) that can acousto-optically steer the beam.

In the example, the pulses create any suitable number (e.g., two, three, four, or more) of cavitation bubbles 8. Cavitation bubbles 8 may be formed any suitable spatial and temporal distance apart that allows bubbles 8 to interact (e.g., come into contact) with each other. The spatial pulse separation may be selected according to the bubble diameter, which may be 150 micrometers (µm) to 2 millimeters (mm), depending on pulse energy. For example, a 10 microjoule (µJ) pulse may yield a 150 to 300 µm diameter; a 6 µJ pulse may yield a 254 µm diameter; and 1 mJ pulse may yield a 1 mm diameter. If a scanner forms cavitation bubbles 8, the scan rate (which determines the temporal separation) may be selected according to the lifetime of bubbles to yield bubbles that are sufficiently temporally close to interact. For example, the bubble lifetime may be approximately 0.1 to 0.3 milliseconds (ms). A scan rate of 50 kilo hertz (kHz) forms bubbles every 1/50 kHz = 20 microseconds (µs), so neighboring bubbles are inflated long enough to interact.

Computer 7 controls components of system 2 in accordance with computer programs. For example, computer 7 instructs laser device 4 and multiplexer / scanner 6 focus laser pulses at the vitreous to create a bubble jet to fragment a floater or remove floater fragments.

### 1.1 Laser-Slit Lamp System

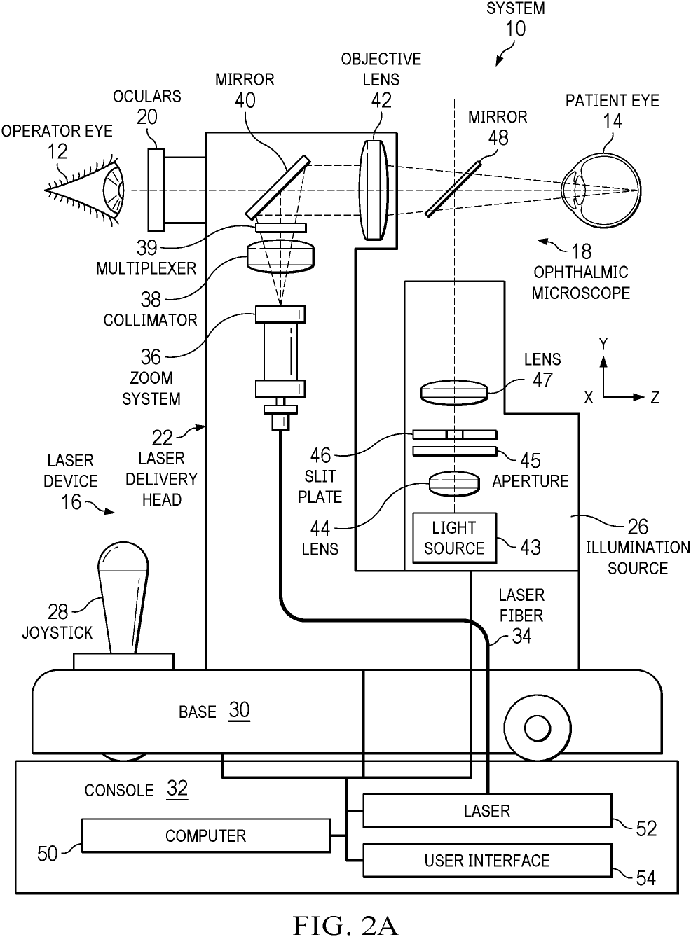

FIGS. 2A and 2B illustrate an example of an ophthalmic laser system 10 with a beam multiplexer that can create cavitation bubbles to form a bubble jet, according to certain embodiments. FIG. 2A illustrates an example of ophthalmic laser system 10 with a beam multiplexer. FIG. 2B illustrates an example of a beam multiplexer comprising a beam doubler 60 that may be used in system 10 of FIG. 2A.

In the example, ophthalmic laser system 10 allows an operator (with an operator eye 12) to see a floater within a patient eye 14. Ophthalmic laser system 10 comprises oculars 20, a laser delivery head 22, a slit illumination source 26, a positioning device (such as a joystick 28), a base 30, and a console 32, coupled as shown. Laser delivery head 22 includes a laser fiber 34, a zoom system 36, a collimator 38, a beam multiplexer 39, a mirror 40, and an objective lens 42, coupled as shown. Slit illumination source 26 includes a light source 43, condenser lens 44, a variable aperture 45, a variable slit plate 46, a projection lens 47, and a mirror 48, coupled as shown. Console 32 includes a computer 50, a laser 52, and a user interface 54, coupled as shown.

As an overview, ophthalmic laser system 10 includes a laser device 16 (e.g., laser 52, laser fiber 34, and laser delivery head 22) and an ophthalmic microscope 18, which includes a slit lamp (e.g., oculars 20, objective lens 42, mirror 48, and slit illumination source 26). Operator eye 12 utilizes the optical path from oculars 20 through mirror 40, objective lens 42, and mirror 48 to view patient eye 14. A laser beam follows the laser path from laser 52 through laser delivery head 22 and mirror 48 to treat patient eye 14.

According to the overview, laser device 16 directs a laser beam comprising laser pulses towards a floater within eye 14. Ophthalmic microscope 18 gathers light reflected from within eye 14 to yield an image of eye 14. Laser beam multiplexer 39 multiplexes (e.g., splits or otherwise modulates) the laser beam into beams that form a cavitation bubbles in the vitreous, and may be any suitable multiplexer as described with reference to FIG. 1. Computer 50 instructs laser device 16 to direct the laser pulses towards the vitreous to form cavitation bubbles that create a bubble jet.

In more detail, in certain embodiments, oculars 20 allow operator eye 12 to view patient eye 14. Laser delivery head 22 delivers a laser beam of laser pulses from laser 52 through laser fiber 34 to patient eye 14. Laser 52 is any suitable laser that generates a laser beam as described with reference to FIG. 1. Zoom system 36 changes the spot size of the laser beam that exits fiber 34. Collimator 38 collimates the laser beam, and mirror 40 directs the beam through objective lens 42, which focuses the beam. Zoom system 36 and collimator 38 direct a parallel laser beam to mirror 40 to focus the laser beam onto the image plane of ophthalmic microscope 18. Mirror 40 may be a dichroic mirror that is reflective for the laser beam wavelength and transmissive for visible light.

Slit illumination source 26 of laser system 10 provides light that illuminates the surgical site of patient eye 14. Slit illumination source 26 includes light source 43, which emits light such as a high-intensity illumination light. Condenser lens 44 directs the light towards variable aperture 45 and variable slit plate 46. Variable aperture 45 defines the height of the light in the y-direction, and variable slit plate 43 defines the width of the light in the x-direction to form the light into a slit shape. Projection lens 47 directions the light towards prism mirror 48, which directs the slit of light into patient eye 14.

Base 30 supports laser delivery head 22 and slit illumination source 26. Joystick 28 moves base 30. Console 32 includes components that support the operation of system 10. Computer 50 of console 32 controls of the operation of components of system 10, e.g., base 30, laser delivery head 22, slit illumination source 26, laser 52, and/or user interface 54. User interface 54 communicates information between the operator and system 10.

FIG. 2B illustrates an example of a beam multiplexer comprising a beam doubler 60. Beam doubler 60 splits a laser beam into a plurality of beams, which are directed to objective lens 42. At objective lens 42, beams have different intensities I1, I2, which yield cavitation bubbles 8 (8a, 8b) of different diameters. In the example, intensity I1 is greater than intensity I2 and yields a bubble 8a with a greater diameter than that of bubble 8b. As described above, the direction of motion of the bubble jet is towards the smaller bubble. In other examples, cavitation bubbles 8 may have substantially the same diameter.

In the example, beam doubler 60 includes a wave plate 62, e.g., a half-wave plate, and a prism 64, e.g., a Wollaston prism. Wave plate 62 is an optical device that alters the polarization state of a light wave travelling through it. In the example, a half-wave plate shifts the polarization direction of linearly polarized light. Prism 64 is a transparent optical device with flat surfaces that refract light. At least one surface is angled (not parallel) to another surface. A Wollaston prism separates light into two orthogonally linearly polarized beams that yield two bubbles. Prism 64 may have any suitable separation, e.g., 0.2 to 5.0 degrees, to yield bubbles with any suitable bubble center separation, e.g., 0.1 to 3.0 millimeters (mm). For example, 2 millijoule (mJ) laser pulses yield bubbles with diameters of around 1.8 mm. A prism separation of 0.5 degrees results in a bubble center separation of 160 mm*sin (0.5) = 1.4 mm, which is close enough to allow the bubbles to interact.

The intensity ratio I₁/I₂ of the bubbles (and thus the relative diameters of the bubbles) can be changed by adjusting, e.g., rotating, wave plate 62 and/or prism 64. That is, wave plate 62 can create cavitation bubble with different intensities and different diameters. Since the direction of the bubble jet is towards the smaller bubble, wave plate 62 may be used to adjust the direction of the bubble jet.

### 1.2 Floater Detection-Laser System

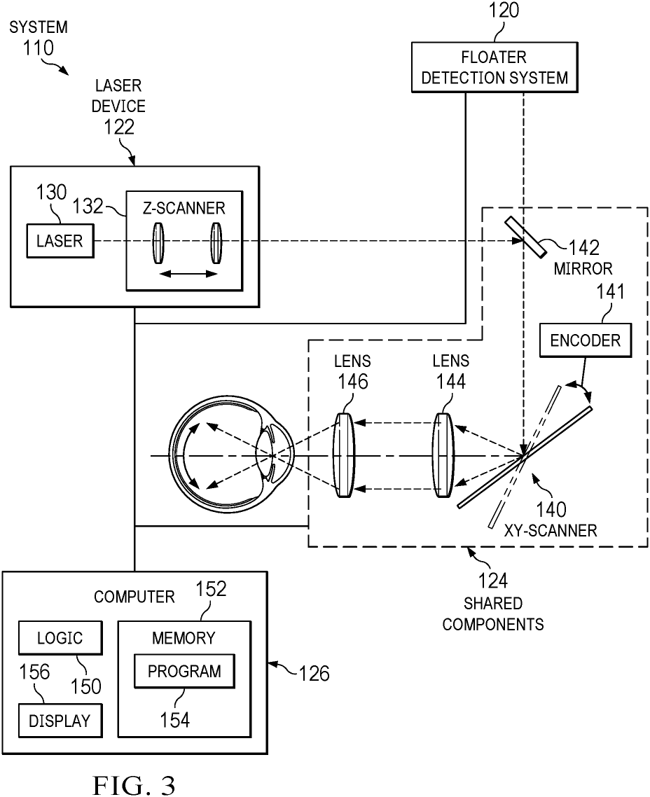

FIG. 3 illustrates an example of an ophthalmic laser surgical system 110 with a scanner that can create cavitation bubbles to form a bubble jet, according to certain embodiments. As an overview, system 110 includes a floater detection system 120, a laser device 122, one or more shared components 124, and a computer 126, coupled as shown. Laser device 122 includes a laser 130 and a z-scanner 132, coupled as shown. Shared components 124 include an xy-scanner 140, an xy-encoder 141, and optical elements (such as a mirror 142 and lenses 144 and 146), coupled as shown. Computer 126 includes logic 150, a memory 152 (which stores a computer program 154), and a display 156, coupled as shown.

As an overview of operation of system 110, floater detection system 120 directs a detection beam along a detection beam path towards an eye and determines the location of the floater. Laser device 122 receives the z-location of the floater relative to the retina from the floater detection system and directs a laser beam along a laser beam path towards the z-location of the floater. Shared component xy-scanner receives the detection beam and directs the detection beam along the detection beam path towards the floater. Xy-scanner 140 also receives the laser beam from the laser device and directs the laser beam along the same detection beam path towards the floater.

Turning to the parts of the system, floater detection system 120 includes one or more detection devices that detect a floater in an eye. To detect a floater, a detection device directs a detection beam towards the eye, detects the beam reflected from the eye, and detects the floater using the reflected beam. The device may detect the floater from the reflected beam by sensing a change in the beam that indicates the presence of a floater or by generating an image of the floater or the floater’s shadow on the retina (“floater shadow”), which may be displayed on display 156. The devices may utilize the same or different technologies, e.g., scanning laser ophthalmoscopy (SLO) and/or optical coherence tomography (OCT). One or more detection devices may provide the x, y, and/or z locations of the floater.

Laser device 122 includes laser 130, which generates a laser beam comprising laser pulses. Laser 130 may comprise any suitable laser as described with reference to FIG. 1, e.g., a femtosecond laser. Z-scanner 132 longitudinally directs the focal point of the laser beam to a specific location in the z-direction. In certain embodiments, laser device 122 includes a multiplexer that multiplexes a laser beam to yield multiple cavitation bubbles that create a bubble jet. The multiplexer may be any suitable multiplexer as described with reference to FIG. 1.

Shared components 124 direct detection and laser beams from floater detection system 120 and laser device 122, respectively, towards the eye. Because detection and laser beams both use shared components 124, both beams are affected by the same optical distortions. Accordingly, when the detection beam is used to aim the laser beam, the distortions are canceled out, which improves the accuracy of the laser beam. As an example of operation, mirror 142 directs a beam towards xy-scanner 140, which transversely directs the focal point of the laser beam in the x- and y-directions towards lens 144. Xy-scanner 140 may comprise any suitable scanner as described with reference to FIG. 1. Lenses 144 and 146 direct the beam towards eye. Xy-encoder 141 detects the position of xy-scanner 140 and reports the position in encoder units to floater detection system 120, laser device 122, and/or computer 26. Shared components 124 may also provide spectral and polarization coupling and decoupling of detection and laser beams to allow the beams to share the same path.

Computer 126 controls components of system 110 in accordance with computer program 154. For example, computer 126 controls components (e.g., floater detection system 120, laser device 122, and shared components 124) to detect a floater and focus a laser beam at the floater. Computer 126 may be separated from components or may be distributed among system 110 in any suitable manner, e.g., within floater detection system 120, laser device 122, and/or shared components 124. In certain embodiments, portions of computer 126 that control floater detection system 120, laser device 122, and/or shared components 124 may be part of floater detection system 120, laser device 122, and/or shared components 124, respectively.

### 2. Floaters

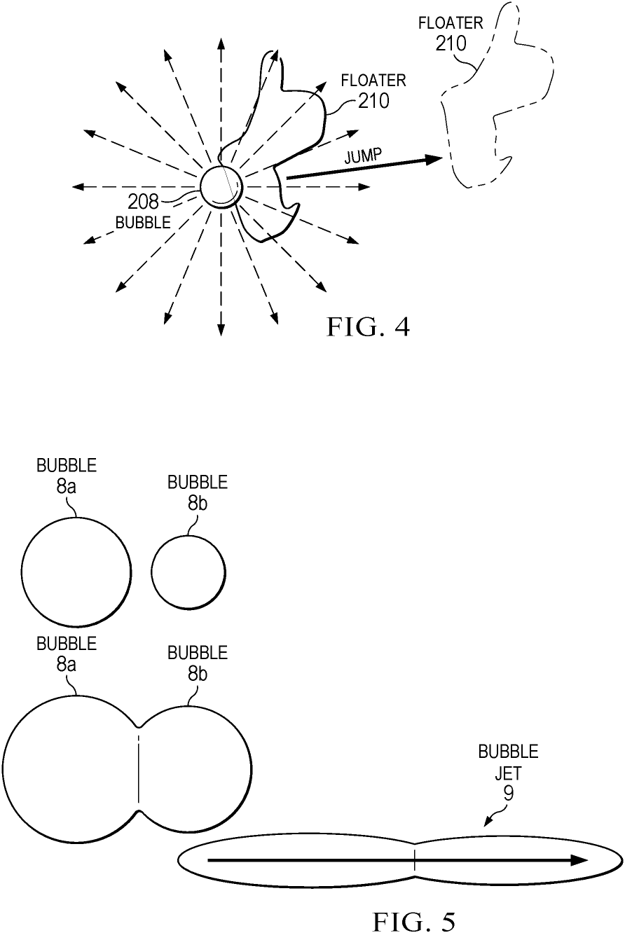

FIG. 4 illustrates an example of a laser pulse causing a floater 210 to jump. If the pulse hits the center of floater 210, the bubble fragments floater 210. However, if the pulse hits the periphery of floater 210, the bubble rapidly pushes floater 210, causing it to jump. If floater 210 jumps a distance of, e.g., 1 millimeter (mm), the laser will have to be redirected with the positioning device.

In certain embodiments, system 2 may create a laser pulse pattern that reduces the likelihood of causing a floater to jump. The pattern places pulses in the path where floater 210 could jump (e.g., outside the area of floater 210) in order to limit the movement of floater 210. That is, the coverage of the pulse pattern (i.e., the area enclosed by the outermost pulses of the pulse pattern) may be substantially centered about the centroid of floater 210 and may be larger than at least a majority of floater 210.

### 3. Bubble Jets

FIG. 5 illustrates an example of a bubble jet 9 that may be created by ophthalmic laser system 10 and 110 of FIGS. 2A, 2B, and 3. In the example, ophthalmic laser system 10 forms cavitation bubbles 8 that create bubble jet 9. For example, a low repetition rate (e.g., less than 3 pulses per second (pps)) laser device with a beam multiplexer may form bubble jet 9. Cavitation bubbles 8 include a larger bubble 8a and a smaller bubble 8b. The direction of motion of bubble jet 9 is towards smaller bubble 8b. The direction may be determined by a line drawn through the centers of bubbles 8, from the larger bubble 8a towards the smaller bubble 8b.

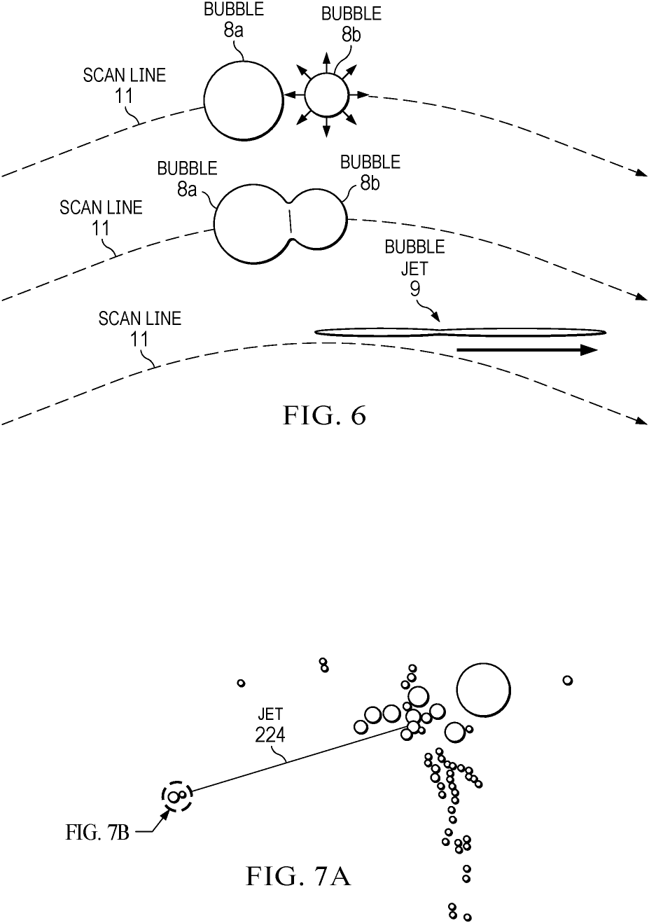

FIG. 6 illustrates an example of a bubble jet 9 that may be created by ophthalmic laser system 10 and 110 of FIGS. 2A, 2B, and 3. In the example, ophthalmic laser system 10 creates cavitation bubbles 8 (8a, 8b) along a scan line 11 indicating where the scanner scans. Bubble 8b is created after bubble 8a at a distance where bubbles 8 can coalesce. Cavitation bubbles 8 interact to create a bubble jet 9 that propagates tangentially to the track of scan line 11.

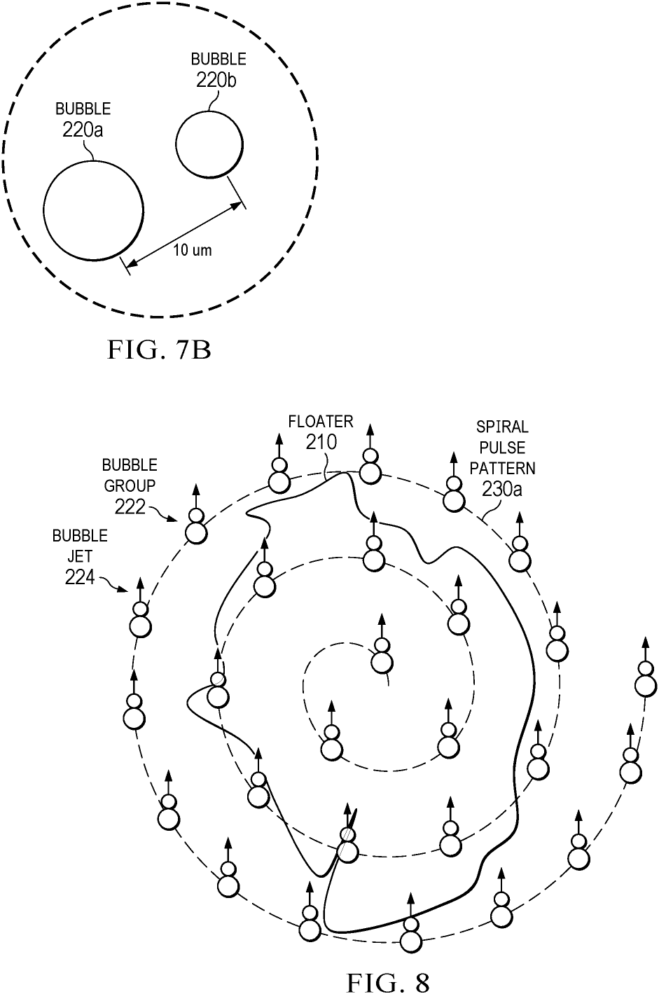

FIGS. 7A and 7B illustrate examples of a bubble jet 224 resulting from cavitation bubbles 220 (220a, 220b) of different diameters, where bubble 220a is larger than bubble 220b. Cavitation bubbles 220 (220a, 220b) maybe formed any suitable distance apart that allows bubbles 220 to interact, e.g., 5 to 20 microns, such as approximately 10 microns apart. Interaction between cavitation bubbles 220a and 220b form bubble jet 224 that flows towards the smaller bubble 220b.

### 4. Pulse Patterns

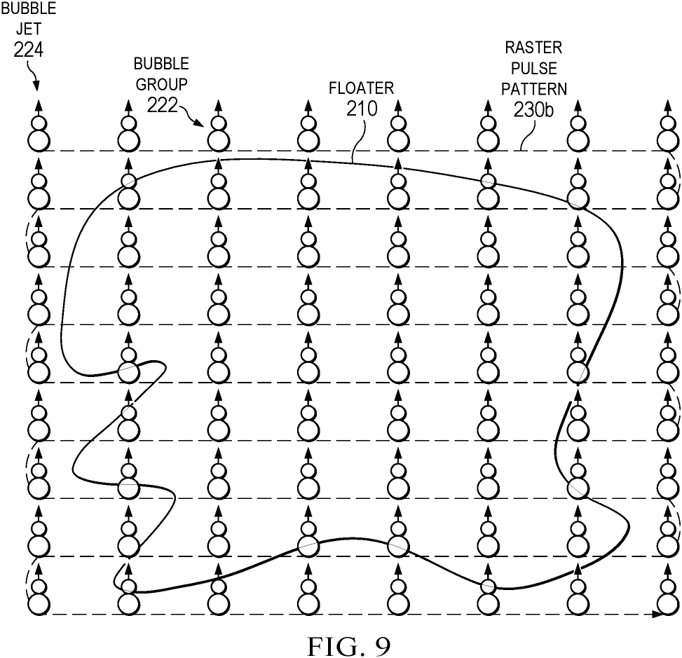

FIGS. 8 and 9 illustrate examples of enface pulse patterns 230 (230a and 230b) that may be generated by system 10 of FIG. 1. Pulse patterns 230 create bubble jets that fragment a floater and/or remove floater fragments. In the examples, pulse patterns 230 include pulse groups, where each pulse group yields a bubble group 222 with cavitation bubbles proximate to each other to create a bubble jet. Pulse patterns 230 may have any suitable size or shape in two-or three-dimensions, and a bubble group 222 may have any suitable number of bubbles. In certain embodiments, the enface coverage of a pattern 230 may cover the enface dimension of the floater. In certain cases (e.g., for a thick floater), multiple enface patterns 230 may be applied at different depths, yielding a three-dimensional pattern 230.

Pulse patterns 230 may be formed in any suitable manner. For example, a medium repetition rate (e.g., 100 Hz to 10 kHz) picosecond or femtosecond laser with a beam multiplexer can create a pulse pattern 230. In the example, the laser pulse energy per spot is 20 µJ, and the corresponding bubble oscillation period is T = 13.3 us*20^{⅓} = 36.1 µs. The repetition rate of 100 to 10 kHz corresponds to a pulse separation of 100 to 10,000 µs. In this example, the previous bubble group 222 disappears before the next pulse group arrives, so there is no interaction between the pulse group and the remains of the previous bubble group 222.

As another example, a high repetition rate (e.g., 40 to 150 kHz) picosecond or femtosecond laser with a beam multiplexer can create a pulse pattern 230. In the example, the pulse energy is 20 µJ per spot, the repetition rate is 40 kHz, and the pulse separation time is 25 µs. Thus, the next pulse group arrives when the previous bubble group 222 (or re-bouncing bubbles) still exist (or are living or alive). Under these conditions, different bubble groups 222 interact to yield a multi-group interaction, e.g., two groups of two bubbles yield a four-bubble interaction. The multi-group interaction creates bubbles jets to fragment a floater and/or remove floater fragments.

As another example, a high repetition rate (e.g., 40 to 150 kHz) laser creates a pulse pattern 230. In the example, pulse pattern is a spiral scan that starts at the center of the visual field to fragment a floater and move the floater fragments away from the visual field. The spiral has a large (e.g., 50 um) tangential spot separation, the laser pulse energy per spot is 20 µJ, and the corresponding bubble oscillation period is T = 13.3 us*20^{⅓} = 36.1 µs. The repetition rate of 40 to 150 kHz corresponds to a pulse separation of 6.67 to 25 us. Thus, the next pulse arrives when the previous cavitation bubble still exists to form a bubble jet. The direction of the jet is tangential to the spiral, and the length of the jet may be as long as several millimeters.

### 4.1 Spiral Pulse Patterns

FIG. 8 illustrates an example of a spiral pulse pattern 230a. Spiral pulse pattern 230a includes a spiral pattern of pulse groups that yield bubble groups 222, where each bubble group 222 is designed to yield a bubble jet 224. In the example, bubble jets 224 are created with jets pointing in the same direction to optimize the kinetic energy of bubble jets 224. Spiral pulse pattern 230a may be created with any suitable number of pulses (e.g., 10 to 1000 pulses), tangential spot separation (e.g., 2 to 100 µm), and radial spot separation (e.g., 2 to 200 µm).

### 4.2 Raster Pulse Patterns

FIG. 9 illustrates an example of a raster pulse pattern 230b. Raster pulse pattern 230b includes a raster pattern of pulse groups that yield bubble groups 222, where each bubble group 222 is designed to yield a bubble jet 224. In the example, bubble jets 224 are created with jets pointing in the same direction to optimize the kinetic energy of bubble jets 224. The raster pattern is formed by scanning in one direction to form a row of pulses, turning around at the end of the row, and then scanning in the opposite direction proximate to the previous row to form the next row of pulses. Raster pulse pattern 230b may be created with any suitable number of pulses (e.g., 10 to 1000 pulses), spot separation in the same row (e.g., 2 to 100 µm), and row separation (e.g., 2 to 200 µm).

### 5. Example Methods

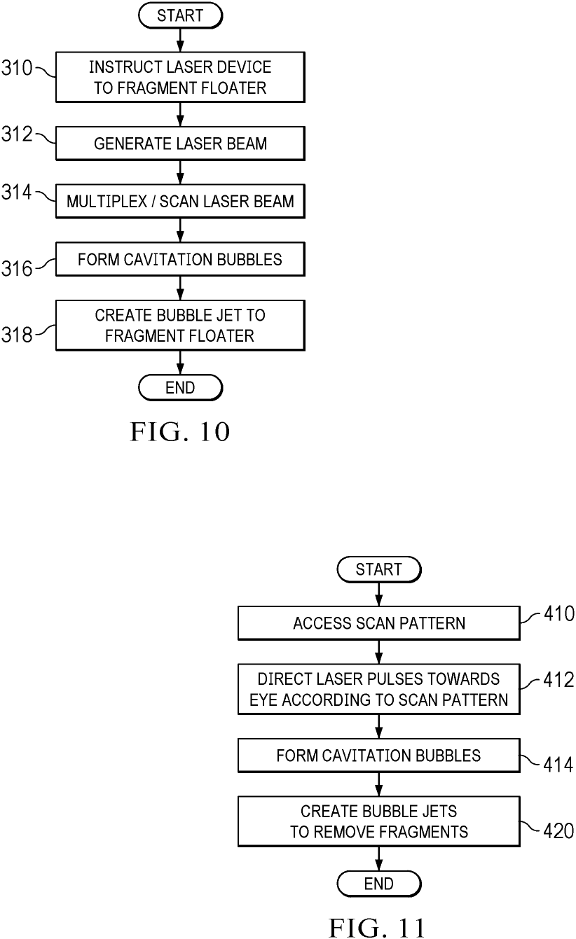

FIG. 10 illustrates an example of a method for creating a bubble jet to fragment a floater, which may be performed by system 10 of FIG. 1. The method starts at step 310, where a computer instructs a laser device to fragment the floater. The laser device generates a laser beam at step 312. The laser beam may comprise laser pulses such as femtosecond pulses. The laser beam is multiplexed and/or scanned at step 314 to yield multiple cavitation bubbles in the vitreous.

The laser pulses form cavitation bubbles at step 316 to create a bubble jet. The bubbles may have different (or the same) diameters. In certain embodiments, the cavitation bubbles are arranged to direct the bubble jet in a particular direction, e.g., in the direction of the smaller bubble. The cavitation bubbles create the bubble jet at step 318 to fragment the floater.

FIG. 11 illustrates an example of a method for creating bubble jets to remove floater fragments, which may be performed by system 10 of FIG. 1. The method starts at step 410, where a computer accesses a pulse pattern for a laser device. The pulse pattern may be designed to control the direction of the floater fragment removal. The computer instructs the laser device to direct laser pulses towards a floater according to the pulse pattern at step 412.

The pulses form cavitation bubbles at step 414. In certain embodiments, the cavitation bubbles are arranged to create bubble jets that point in one or more directions that facilitate removal of the fragments. The cavitation bubbles create bubble jets to remove floater fragments at step 420. The forces of the bubble jets move the fragments away from the visual axis. In addition, after some bubbles collapse, longer-living gas bubbles become entangled in the fragments and move them away from the visual axis.

A component (such as a control computer) of the systems and apparatuses disclosed herein may include an interface, logic, and/or memory, any of which may include computer hardware and/or software. An interface can receive input to the component and/or send output from the component, and is typically used to exchange information between, e.g., software, hardware, peripheral devices, users, and combinations of these. A user interface is a type of interface that a user can utilize to communicate with (e.g., send input to and/or receive output from) a computer. Examples of user interfaces include a display, Graphical User Interface (GUI), touchscreen, keyboard, mouse, gesture sensor, microphone, and speakers.

Logic can perform operations of the component. Logic may include one or more electronic devices that process data, e.g., execute instructions to generate output from input. Examples of such an electronic device include a computer, processor, microprocessor (e.g., a Central Processing Unit (CPU)), and computer chip. Logic may include computer software that encodes instructions capable of being executed by an electronic device to perform operations. Examples of computer software include a computer program, application, and operating system.

A memory can store information and may comprise tangible, computer-readable, and/or computer-executable storage medium. Examples of memory include computer memory (e.g., Random Access Memory (RAM) or Read Only Memory (ROM)), mass storage media (e.g., a hard disk), removable storage media (e.g., a Compact Disk (CD) or Digital Video or Versatile Disk (DVD)), database, network storage (e.g., a server), and/or other computer-readable media. Particular embodiments may be directed to memory encoded with computer software.

Although this disclosure has been described in terms of certain embodiments, modifications (such as changes, substitutions, additions, omissions, and/or other modifications) of the embodiments will be apparent to those skilled in the art. Accordingly, modifications may be made to the embodiments without departing from the scope of the invention. For example, modifications may be made to the systems and apparatuses disclosed herein. The components of the systems and apparatuses may be integrated or separated, or the operations of the systems and apparatuses may be performed by more, fewer, or other components, as apparent to those skilled in the art. As another example, modifications may be made to the methods disclosed herein. The methods may include more, fewer, or other steps, and the steps may be performed in any suitable order, as apparent to those skilled in the art.

To aid the Patent Office and readers in interpreting the claims, Applicants note that they do not intend any of the claims or claim elements to invoke 35 U.S.C. §112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the applicants to refer to structures known to those skilled in the relevant art and is not intended to invoke 35 U.S.C. §112(f).

What is claimed:

1. An ophthalmic laser system for treating a floater in a vitreous of an eye, comprising:
a laser device configured to direct a plurality of laser pulses towards the floater in the vitreous of the eye, the laser device comprising:
a laser configured to generate a laser beam; and
a beam multiplexer configured to split the laser beam into a plurality of beams that form a plurality of cavitation bubbles to create a bubble jet in the vitreous of the eye;
an ophthalmic microscope configured to provide an image of a shadow cast by the floater onto a retina of the eye; and
a computer configured to:
instruct the laser device to direct the plurality of beams towards the floater in the vitreous in order to create the bubble jet to treat the floater.

2. The ophthalmic laser system of claim 1, the beam multiplexer comprising an optical device selected from the following: a diffractive optical element (DOE), a holographic optical element (HOE), a spatial light modulator (SLM), a polarizing prism, a beam amplitude splitting interferometer, a wavefront splitting interferometer, or a birefringent optical component.

3. The ophthalmic laser system of claim 1, the beam multiplexer comprising:
a wave plate configured to alter a polarization state of the laser beam; and
a prism configured to separate the laser beam into the plurality of beams.

4. The ophthalmic laser system of claim 3:
the wave plate comprising a λ/2 wave plate configured to shift the polarization state of the laser beam; and
the prism comprising a Wollaston prism configured to separate the laser beam into the beams with orthogonal polarization.

5. The ophthalmic laser system of claim 1, the beam multiplexer configured to split the laser beam into the plurality of beams that form the plurality of cavitation bubbles with a bubble center separation of 5 to 20 microns.

6. The ophthalmic laser system of claim 1, the beam multiplexer configured to:
create a first cavitation bubble with a first diameter; and
create a second cavitation bubble with a second diameter, the second diameter different from the first diameter.

7. The ophthalmic laser system of claim 1, the computer configured to instruct the laser device to direct the plurality of beams towards the floater by:
instructing the laser device to direct the plurality of beams to form the plurality of cavitation bubbles arranged to direct the bubble jet in a particular direction.

8. The ophthalmic laser system of claim 1, the computer configured to instruct the laser device to direct the plurality of beams towards the floater by:
instructing the laser device to direct the plurality of beams to form the plurality of cavitation bubbles arranged in a spiral enface pattern.

9. The ophthalmic laser system of claim 1, the computer configured to instruct the laser device to direct the plurality of beams towards the floater by:
instructing the laser device to direct the plurality of beams to form the plurality of cavitation bubbles arranged in a raster enface pattern.

10. A method for treating a floater in a vitreous of an eye, comprising:
generating, by a laser of a laser device, a laser beam;
splitting, by a beam multiplexer of the laser device, the laser beam into a plurality of beams that form a plurality of cavitation bubbles to create a bubble jet in the vitreous of the eye;
providing, by an ophthalmic microscope, an image of a shadow cast by the floater onto a retina of the eye;
instructing, by a computer, the laser device to direct the plurality of beams towards the floater in the vitreous in order to create the bubble jet to treat the floater; and
directing, by the laser device, the plurality of beams towards the floater in the vitreous of the eye.

11. The method of claim 10, the beam multiplexer comprising an optical device selected from the following: a diffractive optical element (DOE), a holographic optical element (HOE), a spatial light modulator (SLM), a polarizing prism, a beam amplitude splitting interferometer, a wavefront splitting interferometer, or a birefringent optical component.

12. The method of claim 10, further comprising:
altering, by a wave plate of the beam multiplexer, a polarization state of the laser beam; and
separating, by a prism of the beam multiplexer, the laser beam into the plurality of beams.

13. The method of claim 12:
the wave plate comprising a λ/2 wave plate configured to shift the polarization state of the laser beam; and
the prism comprising a Wollaston prism configured to separate the laser beam into the beams with orthogonal polarization.

14. The method of claim 10, wherein splitting the laser beam comprises:
splitting the laser beam into the plurality of beams that form the plurality of cavitation bubbles with a bubble center separation of 5 to 20 microns.

15. The method of claim 10, further comprising:
creating, by the beam multiplexer, a first cavitation bubble with a first diameter; and
creating, by the beam multiplexer, a second cavitation bubble with a second diameter, the second diameter different from the first diameter.

16. The method of claim 10, wherein instructing the laser device to direct the plurality of beams towards the floater comprises:
instructing the laser device to direct the plurality of beams to form the plurality of cavitation bubbles arranged to direct the bubble jet in a particular direction.

17. The method of claim 10, wherein instructing the laser device to direct the plurality of beams towards the floater comprises:
instructing the laser device to direct the plurality of beams to form the plurality of cavitation bubbles arranged in a spiral enface pattern.

18. The method of claim 10, wherein instructing the laser device to direct the plurality of beams towards the floater comprises:
instructing the laser device to direct the plurality of beams to form the plurality of cavitation bubbles arranged in a raster enface pattern.
